(19) United States
(12) Patent Application Publication
(10) Pub. No.: US 2014/0199420 A1
(43) Pub. Date: Jul. 17, 2014

(54) USES OF TOONA SINENSIS EXTRACT

(71) Applicant: National Chung Hsing University, Taichung (TW)

(72) Inventor: Deng-Cheng LIU, Taichung (TW)

(73) Assignee: National Chung Hsing University, Taichung (TW)

(21) Appl. No.: 14/147,271

(22) Filed: Jan. 3, 2014

(30) Foreign Application Priority Data
Jan. 17, 2013 (TW) 102101786

(51) Int. Cl. A61K 36/58; A01N 65/26

(52) U.S. Cl. CPC A61K 36/58; A01N 65/26
USPC 424/769

(57) ABSTRACT

The present invention is related to a Toona sinensis extract for antibacterial. The Toona sinensis extract has the ability to inhibit growth or reproduction of bacteria and also to prevent corruption, color change and acidification of foods. So that, it can replace commercial ingredients and be applied to antibacterial, pharmaceutical composition or preservative that can prevent food corruption.

The current application claims a foreign priority to the patent application of Taiwan No. 102101786 filed on Jan. 17, 2013.

### FIELD OF THE INVENTION

The present invention relates to a plant, specially relates to the uses of Toona sinensis extract.

### DESCRIPTION OF THE RELATED ART

Microorganisms are microscopic organisms including five groups of the bacteria, fungi, viruses, algae and protozoa. The number of bacteria is the most among them. Bacteria widely spread in the ground, water or symbiosis with other organism. For example, there are various bacterial species in human body or on human skin. Therefore, bacteria contribute a lot for human livings. To speak further, many bacterial species can be utilized and is good for human livings. Especially under the booming development of biotechnology, bacteria are utilized not only for manufacturing food, making cheese, yoghourt or soy sauce but also cleaning pollution, treating sewage or producing protein. On the other hand, some bacteria are pathogens which will infect human. For example, Staphylococcal aureus and Escherichia coli can cause diarrhea and dehydration etc., Salmonellosis can cause nausea, infection and bacteremia etc., Pseudomonas spp. can cause infection of lower respiratory tract, urethra and wound etc. Fungi including yeast and mildew most grow in the ground or symbiosis with other animals or plants. A part of fungi play an important role to human living, just as bacteria. For example, fungi can digest organic matter to recycle nutrition as food resource, to manufacture various kind of food or to be bio-pesticide. However, fungi are not totally harmless. Pathogenic fungi cause human or animal sick. For example, mildew infection will cause tinea and sweat stain, candida will cause vaginitis and athlete's foot etc. Fungi also result in food corruption, for example, food become colorful and smells because of mildew.

In order to avoid microorganisms infect human, many business persons have developed various kinds of antibacterial to add into cosmetics or foods to inhibit growth or reproduction of bacteria now. There are three kinds of antibacterial agents, an organic antibacterial agent, an inorganic antibacterial agent and a natural antibacterial agent. Herein, the inorganic antibacterial agent includes nitrite, sulfites, sulfur dioxide and nitrate. The organic antibacterial agent includes benzoic acid and benzoate thereof, sorbic acid and sorbate thereof, paraben and lactate etc. The natural antibacterial agent includes ingredients from wild plant. However, either the organic or the inorganic antibacterial agent is made from chemical synthesis. It can inhibit the growth of bacteria effectively, but still have side effect to human body for long term uses or eating. Because of that, the business persons are dedicated to develop the natural antibacterial agent to prevent the harm of it to human health. Therefore, it becomes popular to find the natural antibacterial agents from plants.

Toona sinensis, also known as Cedrela sinensis, belongs to the class Magnoliopsida in Meliaceae family and its common name is called Chinese toon or Chinese Mahogany. Toona sinensis is a tropical or sub-tropical upland tree and is widely distributed in higher altitude eastwards from India, Nepal, China, Burma, Thailand and Malaysia. It is known that the leaves of Toona sinensis have been used for treating of enteritis, dysentery, metabolic disease, infection, itch, halitosis, vomiting and lack of appetite in traditional Chinese medicine, because of their effects on detoxification and anti-inflammation. In addition, every part of Toona sinensis can be used for treatment. For example, the bark can be used as an astringent and depurative substance, the root powder can be used as a corrective and the fruits can be used as an astringent and treatment eye infection. According to the foresaid, Toona sinensis not only has many cures for human body but also has no side effect. Therefore, it has potential to develop and utilize into many different products.

### SUMMARY OF THE INVENTION

The present invention is related to Toona sinensis extracts for antibacterial. The major propose of the present invention is providing an antibacterial agent comprising a Toona sinensis extract, wherein the Toona sinensis extract is extracted from the leaves of Toona sinensis with a solvent. Furthermore, in the embodiments of the present invention, the solvent is water or an organic solvent, especially ethanol.

The second propose of the present invention is providing a food preservative comprising a Toona sinensis extract, wherein the Toona sinensis extract is extracted from the leaves of Toona sinensis with a solvent. Furthermore, in the embodiments of the present invention, the solvent is water or an organic solvent, especially ethanol.

The third propose of the present invention is providing a method for treating bacterial infection, comprising administering a subject a pharmaceutical composition in an effective amount, wherein the pharmaceutical composition including a Toona sinensis extract, wherein the Toona sinensis extract is extracted from the leaves of Toona sinensis with a solvent. Furthermore, the solvent is water or an organic solvent, especially ethanol.

Moreover, the bacterial infection is an infection by a bacteria, wherein the bacteria is Staphylococcus aureus, Escherichia coli, Pseudomonas, lactobacillus, Salmonella typhimurium, bacillus, Pseudomonas aeroginosa, Vibrio vulnificus, mildew, yeast, Malassezia furfur, Candida albicans, streptococcus, Diphtheroid bacillus, dermatophyte, Helicobacter pylori, clostridium, Lactobacillus acidophilus, staphylococcus, propionibacterium and bacilli.

### BRIEF DESCRIPTION OF THE DRAWINGS

FIG. 1. The changes in total plate count of Taiwanese style sausage treated with different ingredients.

FIG. 2. The changes in anaerobic plate count of Taiwanese style sausage treated with different ingredients.

FIG. 3. The changes in lactic acid bacteria count of Taiwanese style sausage treated with different ingredients.

FIG. 4. The changes in mold and yeast count of Taiwanese style sausage treated with different ingredients.

FIG. 5. The changes in pH value of Taiwanese style sausage with different ingredients.

FIG. 6. The changes in volatile basic nitrogen (VBN) value of Taiwanese style sausage treated with different ingredients.

### DETAILED DESCRIPTION OF THE INVENTION

This present invention discloses a Toona sinensis extract which can inhibit the growth or reproduction of microorganism. Therefore, the Toona sinensis extract can be one of the ingredients of an antibacterial agent, a food preservative or a pharmaceutical composition.

Furthermore, the antibacterial agent can be used directly on an organism or an organism-contacting object with any form to apply it, for example stray, swap or soak etc. And also, to add the antibacterial agent into commodities for daily use, for example clean wash, shampoo, tooth paste, mouthwash, oral spray, facial spray, period commodities for women or cosmetic etc.

The food preservative can be used on an object for inhibiting growth or reproduction of microorganism, increasing storage period and decreasing the rate of object corruption. Generally speaking, the food preservative often serves as a food additive, for example to add into meat product, seafood product, cake, bread or beverage etc.

The pharmaceutical composition includes the Toona sinensis extract for active ingredient and/or a medical acceptable carrier and/or excipient. The pharmaceutical composition can be used as antibiotics for treating infections caused by microorganism. Furthermore, the pharmaceutical composition is in a suitable dosage form of, but not limit to, pill, powder, liquid, tablet, gel etc.

The present invention is further described with the following examples and figures.

### Example 1

### Extraction of Toona sinensis Extract

The fresh leaves of Toona sinensis were dried in an oven at 55° C. for 8 hours. After smashing the dried leaves, 3% weight volume percentage of Toona sinensis water solution were prepared by adding the ground leaves in the distilled water with 120 rpm shaking at 50° C. for 3 hours, and then filtrated the solution through a filter paper. The filtrate, the Toona sinensis water extract, was collected and freeze-dried to powders.

The same as the foresaid process, 3% weight volume percentage of Toona sinensis ethanol solution were prepared by adding the ground leaves in 50% ethanol with 120 rpm shaking at room temperature for 24 hours, and then filtrated through a filter paper. The filtrate, the Toona sinensis ethanol extract, was collected and freeze-dried to powders.

7 grams of the Toona sinensis water extract and the Toona sinensis ethanol extract were separately re-dissolved with 100 mL sterilized water, and then the extracts were thoroughly mixed to get a water solution of Toona sinensis water extract and a water solution of Toona sinensis ethanol extract for coming examples.

### Example 2

### Bacterial Strains

Staphylococcus aureus BCRC 11863, Escherichia coli BCRC 11509 and Salmonella typhimurium BCRC 10905 were obtained from the Department of Veterinary Science, Faculty of Veterinary Medicine, National Chung Hsing University, Taiwan. Pseudomonas aeroginosa BCRC 11864 and Lactobacillus acidophilus ATCC 4356 were purchased from Food Industry Research and Development Institute, Taiwan.

100 μL of S. aureus, E. coli, S. typhimurium and P. aeroginosa were separated inoculated in 2 mL tryptic soy broth (TSB), and shook at 120 rpm, 37° C. for 16˜18 hours. 10 μL of each cultured was streaked on tryptic soy agar plate (TSA plate) and incubated at 37° C. for 16˜18 hours.

L. acidophilus was inoculated in 2 mL MRS broth then shook at 120 rpm, 37° C. for 16˜18 hours. After that, 10 μL of cultured was streaked on MRS agar, the plate was incubated at 37° C. for 16˜18 hour.

### Example 3

### Antibacterial Activity of Toona sinensis Extracts

This example was to add different observed objects with different doses into culture mediums containing different bacterial strains from each other, and then evaluated the diameter of inhibition zone in each culture medium to analyze the antibacterial activity of each observed object in specific dose. Therein, observed objects were the water solution of Toona sinensis water extract and the water solution of Toona sinensis ethanol extract prepared from example 1. Each of the water solution of Toona sinensis extracts were separately added into different culture mediums with 125, 150, 175 and 200 μL. The bacterial strain was selected from the Staphylococcus aureus, Escherichia coli, Salmonella typhimurium, Pseudomonas aeroginosa and Lactobacillus acidophilus prepared from example 2.

In particular, each culture medium was prepared from following procedure. 18 mL of TSA or MRS agar was poured into 9 cm Petri dishes. Solidified TSA agar was drilled with a sterile tube with 1.2 cm diameters under aseptic condition, and then spread 100 μL of bacterial suspension on the agar. Subsequently, separately filled wells with different doses of each observed object. Therein, the bacterial suspension of Staphylococcus aureus, Escherichia coli, Salmonella typhimurium, Pseudomonas aeroginosa was separately spread on TSA agar and incubated at 37° C. for 16˜18 hours and the bacterial suspension of Lactobacillus acidophilus was spread on MRS agar and incubated at 37° C. for 48 hours in an anaerobic jar.

After the foresaid procedure, results of measuring the size of each inhibitory zone in culture medium were shown as Table 1 and 2.

TABLE 1
Antibacterial activity of the Toona sinensis
water extract with different doses

| | Dose of the Toona sinensis water extract (μL) | | | |
|---|---|---|---|---|
| | 125 | 150 | 175 | 200 |
| Bacteria | Diameter of inhibitory zone (cm) | | | |
| Staphylococcus | 0.58 ± 0.02 | 0.58 ± 0.02 | 0.67 ± 0.02 | 0.72 ± 0.02 |
| aureus | | | | |
| Lactobacillus | 0.17 ± 0.03 | 0.23 ± 0.03 | 0.28 ± 0.02 | 0.38 ± 0.02 |
| acidophilus | | | | |

TABLE 2
Antibacterial activity of the Toona sinensis
ethanol extract with different doses

| | Dose of the Toona sinensis ethanol extract (μL) | | | |
|---|---|---|---|---|
| | 125 | 150 | 175 | 200 |
| Bacteria | Diameter of inhibitory zone (cm) | | | |
| Salmonella | 0.87 ± 0.03 | 0.92 ± 0.02 | 1.00 ± 0.06 | 1.03 ± 0.03 |
| typhimurium | | | | |
| Staphylococcus | 1.07 ± 0.02 | 1.12 ± 0.02 | 1.18 ± 0.02 | 1.23 ± 0.03 |
| aureus | | | | |
| Escherichia | 0.97 ± 0.03 | 1.05 ± 0.03 | 1.08 ± 0.02 | 1.13 ± 0.03 |
| coli | | | | |
| Pseudomonas | 0.97 ± 0.02 | 0.98 ± 0.02 | 1.08 ± 0.02 | 1.15 ± 0.03 |
| aeroginosa | | | | |
| Lactobacillus | 0.87 ± 0.03 | 0.97 ± 0.03 | 1.07 ± 0.03 | 1.17 ± 0.03 |
| acidophilus | | | | |

According to the method of measuring the size of each inhibitory zone (Elgayyar et al, 2001), antibacterial activity of each observed object in different doses were analyzed as Table 3, wherein the diameter of inhibitory zone which was larger than 2 cm shows that the observed object had high antibacterial activity and marked as “+++”; the diameter of inhibitory zone which was between 2 to 1 cm shows that the observed object had medium antibacterial activity and marked as “++”; and the diameter of inhibitory zone which was between 1 to 0.6 cm shows that the observed object had low antibacterial activity and marked as “+”.

TABLE 3
Antibacterial activity of the Toona sinensis
extracts with different doses

| | Dose of Toona sinensis extracts (μL) | | | | | | | |
|---|---|---|---|---|---|---|---|---|
| | Toona sinensis | | | | Toona sinensis | | | |
| | water extract | | | | ethanol extract | | | |
| Bacteria | 125 | 150 | 175 | 200 | 125 | 150 | 175 | 200 |
| Salmonella | − | − | − | − | + | + | ++ | ++ |
| typhimurium | | | | | | | | |
| Staphylococcus | − | − | + | + | ++ | ++ | ++ | ++ |
| aureus | | | | | | | | |
| Escherichia | − | − | − | − | + | ++ | ++ | ++ |
| coli | | | | | | | | |
| Pseudomonas | − | − | − | − | + | + | ++ | ++ |
| aeroginosa | | | | | | | | |
| Lactobacillus | − | − | − | − | + | + | ++ | ++ |
| acidophilus | | | | | | | | |

Results of Table 1 and 2 illustrate that different doses of the Toona sinensis water extract and the Toona sinensis ethanol extract can separately generate inhibitory zone to various bacteria. Therefore, the Toona sinensis water extract and the Toona sinensis ethanol extract have effect of inhibiting growth in various bacteria. Furthermore, different doses of the Toona sinensis water extract can generate inhibitory zone to Staphylococcus aureus and Lactobacillus acidophilus, wherein the diameter of inhibitory zone in Staphylococcus aureus was 0.58˜0.72 cm and in Lactobacillus acidophilus was 0.17˜0.38 cm. In addition, different doses of the Toona sinensis ethanol extract generated 0.87˜1.03 cm diameter of inhibitory zone in Staphylococcus aureus, 0.97˜1.15 cm diameter of inhibitory zone in Escherichia coli and 0.87˜1.17 cm diameter of inhibitory zone in Lactobacillus acidophilus.

According to the result of Table 3, different doses of the Toona sinensis ethanol extract still has antibacterial activity and it is better at the doses over 150 μL than at the doses under 150 μL whether adding the Toona sinensis ethanol extract or the Toona sinensis water extract.

### Example 4

### Taiwanese Style Sausage Preparation

By mixing of ground pork ham and diced backfat with the ratio 3:1 and then divided into 5 groups were separately treated with different condition. After treating with different condition, each group was cured at 4° C. for 48 hours and stuffed into hog casings and manually linked in proper length. Then, the each group of sausages was dried in a dry oven at 50˜55° C. for 6 hours. Herein the first group was with no ingredients as negative control, the second group was with 25% sodium lactate as positive control, the third group was with 0.1% potassium sorbate, the forth group was with 125 ppm water solution of the Toona sinensis ethanol extract prepared from example 1 and the fifth group was with 250 ppm water solution of the Toona sinensis ethanol extract prepared from example 1.

### Example 5

### Microbiological Analyses

The each group of sausages prepared from example 4 was packed in equal with vacuum package and stored at 15° C. for 8 weeks. Taking the amounts of sausages from the each group in 0, 1, 2, 3, 4 and 8 weeks were separately packed into sterile bags with sterile water and homogenized with a stomacher (Lab-Blender 400, England) for 30 second to have dilutions of the each group's sausages at different storage times for further microbiological analyzing.

1. Total Plate Count

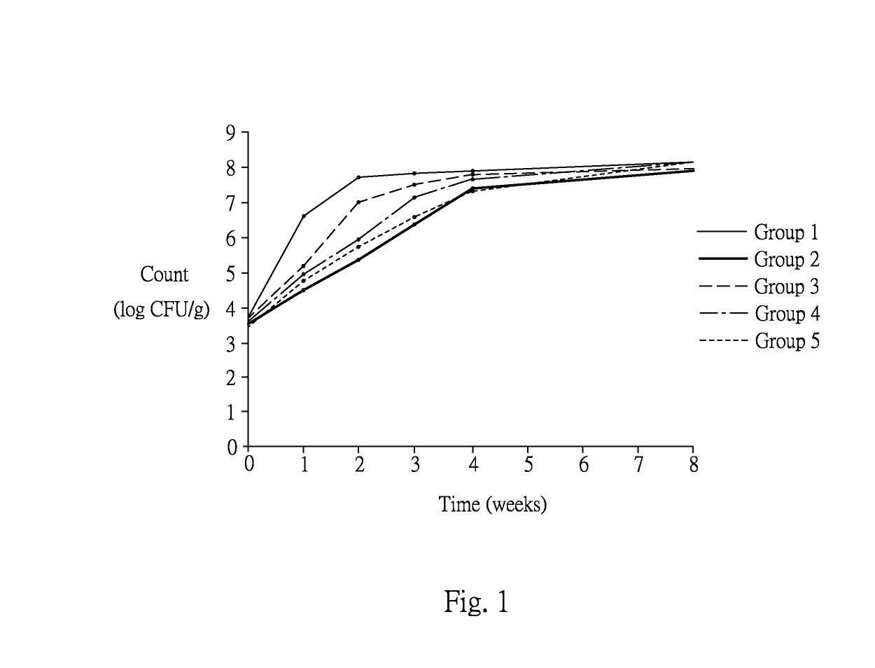

The dilutions of the each group's sausages at different storage times were separately poured into a petri dish with plate count agar, then mixed completely and incubated at 37° C. for 48 hours. After that, the total plate count of each group at each storage time was counted and the results were showed in FIG. 1.

According to FIG. 1, it illustrates that the total plate count in the each group significantly increases with the storage time. Furthermore, the total plate count in the second group counted at each storage time is the least, between 3.51˜7.92 log CFU/g. The total plate counts in the fourth and fifth group, between 3.60˜8.14 log CFU/g and 3.84˜8.06 log CFU/g, are lower than the control group respectively and have no statistical significance with the second group.

Because potassium sorbate and sodium lactate are common preservatives in general food industry and have effect for inhibiting the growth of microorganism. The total plate count in the sausage adding the Toona sinensis ethanol extract is less than that in the sausage without any addition and similar to that in the sausage adding sodium lactate. Therefore, the results show that the present invention of the Toona sinensis ethanol extract can effectively reduce the number of total plate count in food during storage.

2. Anaerobic Plate Count

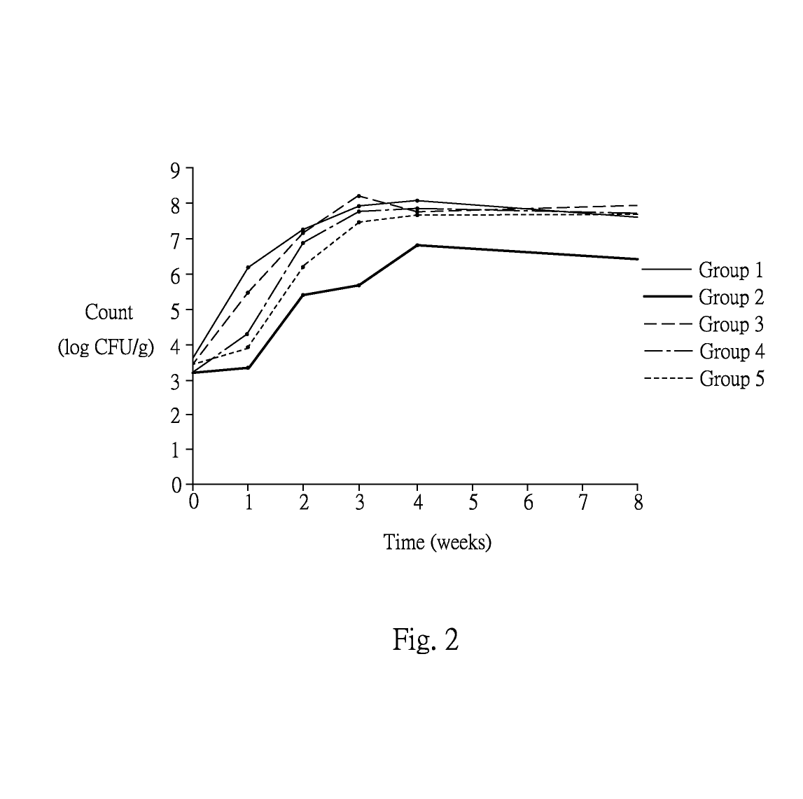

The dilutions of the each group's sausage at different storage times were separately poured into a petri dish containing anaerobic agar, then mixed completely and incubated at 37° C. for 48 hours. After that, the anaerobic plate count of each group at each storage time was counted and the results were showed in FIG. 2.

According to FIG. 2, it shows that the anaerobic plate count in the each group significantly increases with the storage time. The anaerobic plate counts in each group counted in 1, 2, 3, 4 and 8 weeks have significant difference. In addition, the anaerobic plate count in the second group at each storage time, between 3.21˜6.24 log CFH/g, is less than the other groups and the anaerobic plate counts in the fourth and five group at each storage time are separately less than that in the first or third group, wherein the anaerobic plate count in the fifth group is between 3.36˜7.69 log CFU/g which is similar to the anaerobic plate count in the third group.

The results show that the anaerobic plate count at each storage time in the sausage adding the Toona sinensis ethanol extract is not only less than the sausage without any addition but also has better antibacterial effect than the sausage adding potassium sorbate. Moreover, the anaerobic plate count in the sausage adding the Toona sinensis ethanol extract with high doses is similar to that in the sausage adding sodium lactate. Therefore, it shows that the present invention of the Toona sinensis ethanol extract can effectively reduce the anaerobic plate count in food during storage.

3. Lactic Acid Bacteria Count

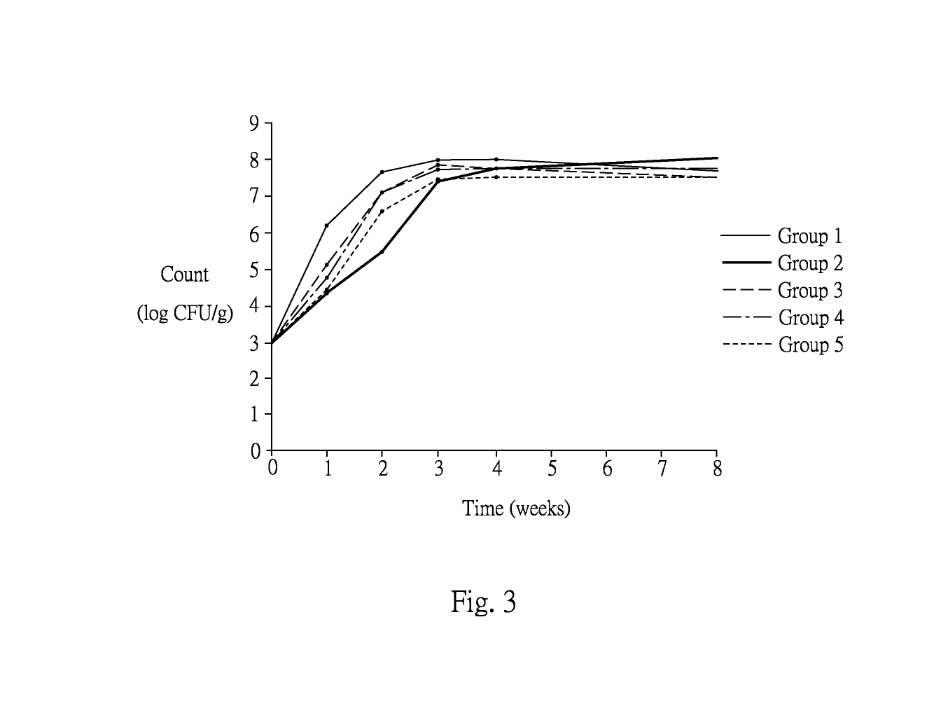

The dilutions of the each group's sausage at different storage times were separately poured into a petri dish, added first layer of MRS agar into the each petri dish and then mixed completely. The second layer of MRS agar was poured on the first layer of MRS agar to produce an anaerobic condition then mixed and incubated at 37° C. for 48 hours in an anaerobic jar. After that, the lactic acid bacteria count of each group at different time was counted and the results were showed in FIG. 3.

The results of FIG. 3 illustrate that the lactic acid bacteria count in the each group significantly increases with the storage time. The lactic acid bacteria counts in the second to fifth group counted at initially four weeks are less than that in the first group respectively. At 4 and 8 weeks, the lactic acid bacteria count in the fifth group is 7.50 log CFU/g which is less than the numbers of lactic acid bacteria count of 7.52 log CFU/g in the second group.

The results show that the lactic acid bacteria count at each storage time in the sausage adding the Toona sinensis ethanol extract is less than that in the sausage without any addition. Moreover, after stored 4 weeks, the lactic acid bacteria count in the sausage adding the Toona sinensis ethanol extract is less than that in the sausage adding sodium lactate or potassium sorbate. In other words, the present invention of the Toona sinensis ethanol extract can inhibit the growth of lactic acid bacteria in food during storage.

4. Mold and Yeast Count

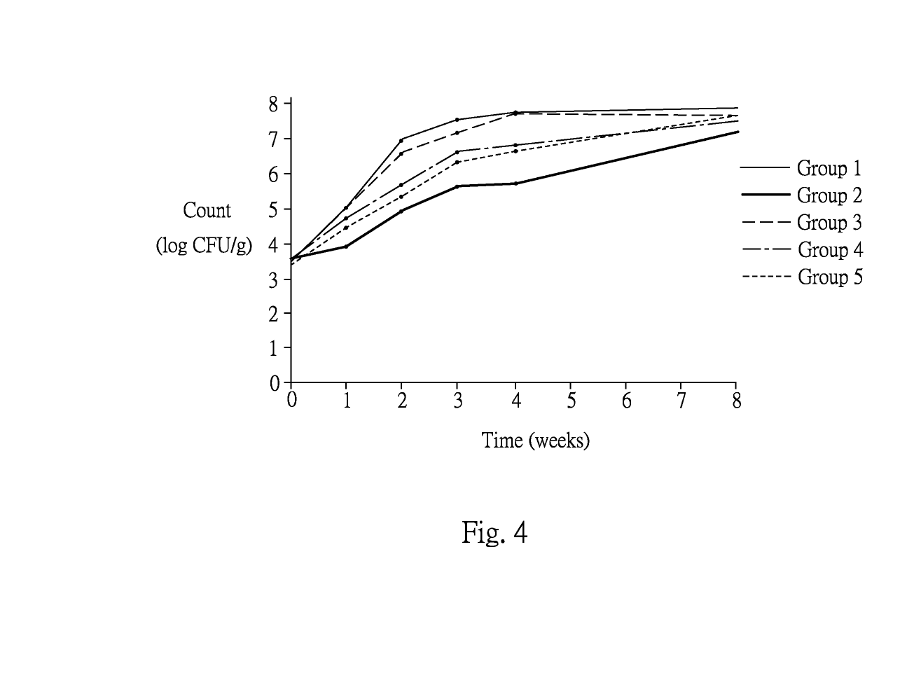

The dilutions of the each group's sausage at different storage times were separately poured into a petri dish containing potato dextrose agar then mixed and incubated at 28° C. for 5 days. After that, the mold and yeast count in each group of sausages at different storage time was counted and the results were showed in FIG. 4.

According to FIG. 4, it illustrates that the mold and yeast count in each group significantly increases with the storage time and it has significant difference in each group between each storage time. Moreover, the mold and yeast counts in the second to fifth group are less than that in the first group from first week respectively. The mold and yeast counts in the fourth and fifth group are less than that in the third group respectively.

The results show that the sausage adding the Toona sinensis ethanol extract can not only reduce the numbers of mold and yeast count but also have better antibacterial effect than the sausage adding potassium sorbate. Therefore, it can be inferred that the present invention of the Toona sinensis ethanol extract can inhibit the growth of mold and yeast in food during storage.

### Experiment 6: pH Values Determination

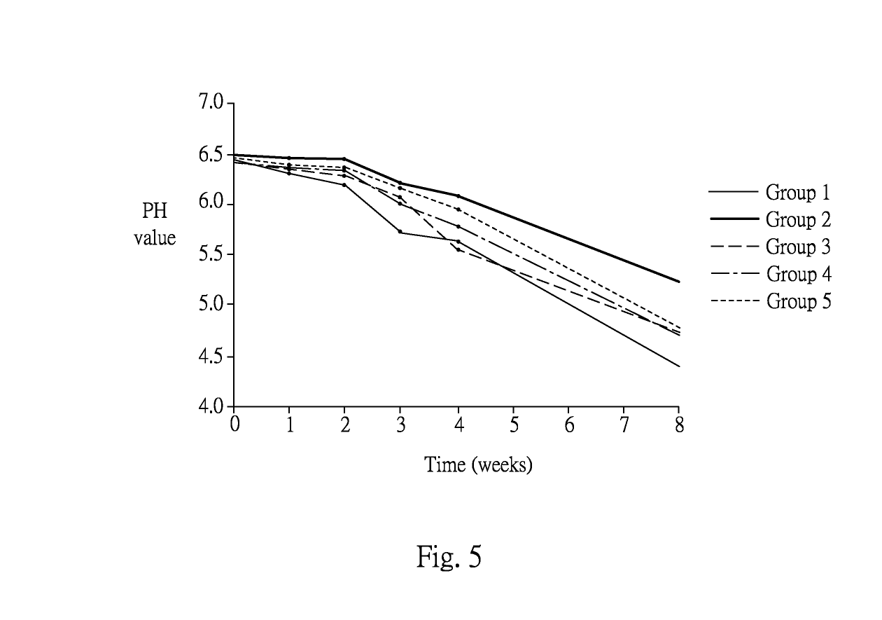

The each group of sausages prepared from example 4 was packed in equal with vacuum package and stored at 15° C. for 8 weeks. The amounts of sausages of the each group in 0, 1, 2, 3, 4 and 8 weeks were separately packed into sterile bags with sterile water and homogenized with a stomacher (Lab-Blender 400, England) for 30 second to have dilutions of each group at different storage time for pH values determination with pH meter (Mettler Teledo 320, Switzerland). The results of pH values in each group at different storage time were showed in FIG. 5.

The results of FIG. 5 illustrate that the pH value in each group significantly decreases with the storage time. The pH values in the second to fifth group are higher than that in the first group at each storage time respectively. After stored 8 weeks, the pH values in the fourth or fifth group are higher than that in the third group.

The results show that the sausage adding the Toona sinensis ethanol extract can decline the process of acidification. And the effect of the decline in the sausage adding the Toona sinensis ethanol extract is better than that adding potassium sorbate. Therefore, the present invention of the Toona sinensis ethanol extract can decline the process of acidification in food during storage.

### Example 7

### The Total Volatile Basic Nitrogen (VBN)

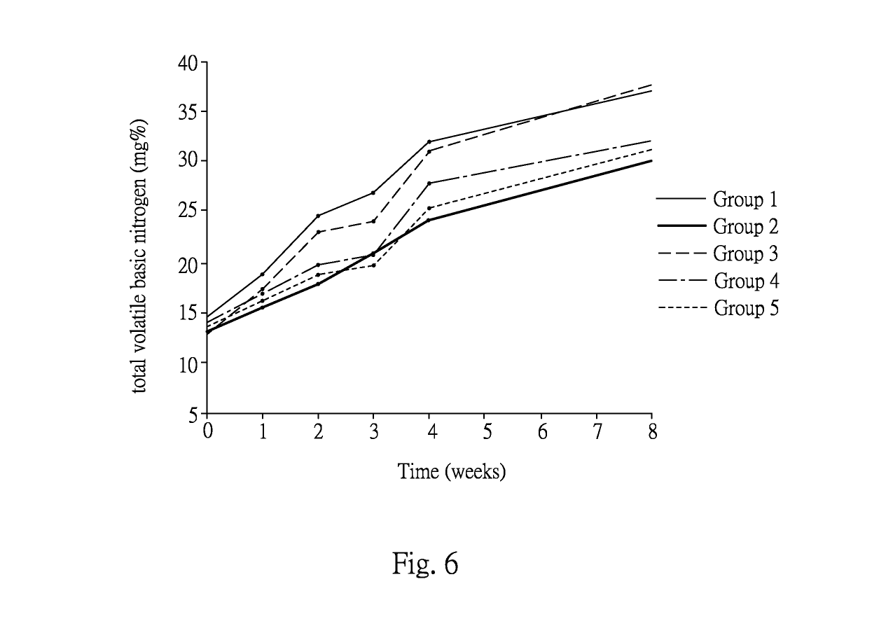

The each group of sausages prepared from example 4 was packed in equal with vacuum package and stored at 15° C. for 8 weeks. Measuring the total volatile basic nitrogen of each group in 0, 1, 2, 3, 4 and 8 weeks was further analyzed the bacteria amount in each group at different storage time. The results were showed in FIG. 6.

The method of analyzing total volatile basic nitrogen descripts as following in detail. There was 5 grams of sausage samples homogenized with 45 mL of 2.2% trichloroacetic acid solution for 15 seconds and centrifuged at 3500 rpm for 5 minutes. The supernatant was filtered and the filtrate was collected. A conway's dish was allowed with 1 mL saturated K₂CO₃ solution and boric acid solution into the inner compartment of the dish, then 1 mL of sample was transferred into the outer compartment of the dish and left at room temperature for 90 minutes. There was 1 mL of 2.2% trichloroacetic acid solution served as blank. The amount of total volatile basic nitrogen was calculated by following formula:

VBN (mg %)=0.014×(A−B)×F×100÷0.1

A: the volume in mL was used during titration for sample

B: the volume in mL was used during titration for blank

F: the titer of HCl

The results of FIG. 6 illustrates that the total volatile basic nitrogen in each group significantly decreases with the storage time. In conclusion, the amounts of total volatile basic nitrogen in the second to fifth group are lower than that in the first group respectively, wherein only in the 8 week, the amount of total volatile basic nitrogen in the third group is higher than that in the first group. Besides, comparing with the first group, the amount of total volatile basic nitrogen in the second, fourth or fifth group is significantly lower.

The results show that the sausage adding the Toona sinensis ethanol extract can decline the amount of total volatile basic nitrogen during storage. Because volatile basic nitrogen is produced from the protein food corruption by microorganism, it indicates that the less microorganism in food, the lower amount of total volatile basic nitrogen. Therefore, it shows that adding the Toona sinensis ethanol extract can effectively inhibit the growth of microorganism in food.

### Example 8

### Sensory Evaluation

The each group of sausages prepared from example 4 was packed in equal with vacuum package and stored at 15° C. for 8 weeks. Taking the amount of sausages from each group in 0, 1, 2, 3, 4 and 8 weeks were cooked. Then, 15˜20 grams of the cooked sausages of each group in different storage time were served immediately to a sensory panel which consisted of 10 meat science-majored faculty. Sensory attributes, including color, off-odor, flavor and overall acceptance were determined using 1˜7 point hedonic scale as the following: “1” is dislike very much or least off-odor, 2 is dislike moderately, 3 is dislike slightly, 4 is neither like nor dislike, 5 is like slightly, 6 is like moderately and 7 is like very much or extremely off-odor. Statistical analysis results were showed in Table 4˜7.

TABLE 4
The changes in color score of sensory evaluation of
every sausage group during storage at 15° C.

| | Storage time (weeks) | | | | | |
|---|---|---|---|---|---|---|
| Group | 0 | 1 | 2 | 3 | 4 | 8 |
| 1 | 5.80 ± 0.20 | 4.40 ± 0.16 | 4.30 ± 0.21 | 3.90 ± 0.28 | 3.30 ± 0.15 | 3.00 ± 0.30 |
| 2 | 5.90 ± 0.23 | 4.70 ± 0.15 | 4.40 ± 0.16 | 4.20 ± 0.25 | 3.90 ± 0.23 | 3.70 ± 0.21 |
| 3 | 5.70 ± 0.21 | 4.70 ± 0.15 | 4.20 ± 0.25 | 4.30 ± 0.21 | 3.50 ± 0.31 | 2.70 ± 0.15 |
| 4 | 5.80 ± 0.20 | 5.10 ± 0.28 | 5.10 ± 0.18 | 4.50 ± 0.17 | 3.70 ± 0.26 | 3.20 ± 0.13 |
| 5 | 5.90 ± 0.23 | 4.90 ± 0.18 | 4.80 ± 0.20 | 4.60 ± 0.22 | 4.00 ± 0.21 | 3.40 ± 0.22 |

TABLE 5
The changes in flavor score of sensory evaluation of
every sausage group during storage at 15° C.

| | Storage time (weeks) | | | | | |
|---|---|---|---|---|---|---|
| Group | 0 | 1 | 2 | 3 | 4 | 8 |
| 1 | 5.70 ± 0.15 | 5.10 ± 0.23 | 4.50 ± 0.22 | 3.60 ± 0.31 | 3.00 ± 0.42 | 2.40 ± 0.34 |
| 2 | 5.70 ± 0.15 | 5.70 ± 0.15 | 4.50 ± 0.34 | 4.10 ± 0.31 | 3.40 ± 0.43 | 3.20 ± 0.13 |
| 3 | 5.50 ± 0.17 | 4.80 ± 0.13 | 3.60 ± 0.27 | 3.70 ± 0.30 | 3.10 ± 0.28 | 2.00 ± 0.15 |
| 4 | 5.40 ± 0.16 | 5.70 ± 0.15 | 4.80 ± 0.25 | 4.20 ± 0.13 | 3.30 ± 0.30 | 3.10 ± 0.18 |
| 5 | 5.40 ± 0.16 | 5.50 ± 0.17 | 4.80 ± 0.29 | 4.60 ± 0.27 | 3.40 ± 0.22 | 3.20 ± 0.20 |

TABLE 6
The changes in off-flavor score of sensory evaluation
of every sausage group during storage at 15° C.

| | Storage time (weeks) | | | | | |
|---|---|---|---|---|---|---|
| Group | 0 | 1 | 2 | 3 | 4 | 8 |
| 1 | 1.40 ± 0.22 | 1.20 ± 0.13 | 1.60 ± 0.22 | 2.50 ± 0.34 | 2.60 ± 0.40 | 3.40 ± 0.37 |
| 2 | 1.10 ± 0.10 | 1.20 ± 0.13 | 1.30 ± 0.15 | 2.20 ± 0.29 | 2.10 ± 0.28 | 2.20 ± 0.42 |
| 3 | 1.50 ± 0.22 | 1.70 ± 0.21 | 2.40 ± 0.37 | 2.40 ± 0.34 | 2.60 ± 0.27 | 4.60 ± 0.45 |
| 4 | 1.20 ± 0.13 | 1.10 ± 0.10 | 1.30 ± 0.15 | 2.10 ± 0.43 | 2.40 ± 0.22 | 2.40 ± 0.22 |
| 5 | 1.10 ± 0.10 | 1.20 ± 0.13 | 1.20 ± 0.13 | 1.60 ± 0.22 | 2.20 ± 0.25 | 2.30 ± 0.26 |

TABLE 7
The changes in overall acceptance of sensory evaluation
of every sausage group during storage at 15° C.

| | Storage time (weeks) | | | | | |
|---|---|---|---|---|---|---|
| Group | 0 | 1 | 2 | 3 | 4 | 8 |
| 1 | 5.90 ± 0.28 | 5.20 ± 0.25 | 4.30 ± 0.30 | 3.30 ± 0.30 | 2.90 ± 0.35 | 2.40 ± 0.43 |
| 2 | 5.90 ± 0.18 | 5.60 ± 0.16 | 4.50 ± 0.37 | 4.00 ± 0.37 | 3.60 ± 0.31 | 3.50 ± 0.31 |
| 3 | 5.40 ± 0.16 | 4.60 ± 0.22 | 3.40 ± 0.31 | 3.70 ± 0.37 | 3.20 ± 0.29 | 1.80 ± 0.20 |
| 4 | 5.80 ± 0.13 | 5.70 ± 0.15 | 5.10 ± 0.28 | 4.10 ± 0.10 | 3.30 ± 0.33 | 3.20 ± 0.20 |
| 5 | 5.70 ± 0.15 | 5.60 ± 0.16 | 5.20 ± 0.29 | 4.40 ± 0.31 | 3.50 ± 0.31 | 3.30 ± 0.15 |

The result of Table 4 illustrates that the color score in each group significantly decreases with the storage time. Therein, the color score in the fourth group maintains 3 between 1˜3 weeks and that in the fifth group maintains 4 between 1˜4 weeks. The result of Table 5 illustrates that the flavor score in each group significantly decreases with the storage time. Therein, the flavor score in the fourth and fifth group can maintain higher than 4 in initially 3 weeks. The result of Table 6 illustrates that the off-flavor score in each group significantly increases according to the storage time. Therein, the off-flavor score in each group has significant difference in 8 weeks. The result of Table 7 illustrates that the overall acceptance score in each group significantly decreases according to the storage time. Therein, the overall acceptance score in the fourth and fifth group maintains 4 in initially 3 weeks.

These results show that the fourth and fifth group adding the Toona sinensis ethanol extract can keep look, favor, and overall acceptance of sausages and be accepted at least 3 weeks stored at 15° C., wherein the fifth group with high dose of the Toona sinensis ethanol extract has better scores, and the overall acceptance scores in fourth and fifth are similar to that in the third group with potassium sorbate. Therefore, this invention of the Toona sinensis ethanol extract has effect just like preservatives that can keep the outlook and favor of foods.

According to foresaid examples, this invention of Toona sinensis extract has the ability to inhibit growth or reproduction of bacteria and also to prevent corruption, color change and acidification of foods. So that, it can be applied for antibacterial or pharmaceutical composition and also preservative to replace commercial ingredients for prevent corruption. In addition, Toona sinensis extract has no effect to human health because of it is extracted from natural plant.

What is claimed is:

1. An antibacterial agent at least includes a Toona sinensis extract.

2. The antibacterial agent according to claim 1, wherein the Toona sinensis extract is extracted from the leaves of Toona sinensis with water.

3. The antibacterial agent according to claim 1, wherein the Toona sinensis extract is extracted from the leaves of Toona sinensis with an organic solvent.

4. The antibacterial agent according to claim 3, wherein the organic solvent is ethanol.

5. A food preservative at least includes a Toona sinensis extract.

6. The food preservative according to claim 5, wherein the Toona sinensis extract is extracted from the leaves of Toona sinensis with an organic solvent.

7. The food preservative according to claim 6, wherein the organic solvent is ethanol.

8. A method for treating bacterial infection, comprising administering a subject a pharmaceutical composition in an effective amount, wherein the pharmaceutical composition including a Toona sinensis extract.

9. The method according to claim 8, wherein the bacterial infection is an infection by a bacteria selected from the group of Staphylococcus aureus, Escherichia coli, Pseudomonas, Lactobacillus acidophilus, Salmonella typhimurium, bacillus, Pseudomonas aeroginosa, Vibrio vulnificus, mildew, yeast, Malassezia furfur, Candida albicans, streptococcus, Diphtheroid bacillus, dermatophyte, Helicobacter pylori, clostridium, lactobacillus, staphylococcus, propionibacterium and bacilli.

10. The method according to claim 8, wherein the Toona sinensis extract is extracted from the leaves of Toona sinensis with water.

11. The method according to claim 8, wherein the Toona sinensis extract is extracted from the leaves of Toona sinensis with an organic solvent.

12. The method according to claim 11, wherein the organic solvent is ethanol.
